(19) United States
(12) Patent Application Publication
(10) Pub. No.: US 2025/0025255 A1
(43) Pub. Date: Jan. 23, 2025

(54) Medical Draping System

(71) Applicant: University of Iowa Research Foundation, Iowa City, IA (US)

(72) Inventor: Bradley Van Voorhis, Iowa City, IA (US)

(73) Assignee: University of Iowa Research Foundation, Iowa City, IA (US)

(21) Appl. No.: 18/774,299

(22) Filed: Jul. 16, 2024

Related U.S. Application Data
(60) Provisional application No. 63/513,982, filed on Jul. 17, 2023.

(51) Int. Cl. A61B 46/00

(52) U.S. Cl. CPC A61B 46/30

(57) ABSTRACT

A medical draping system for enhancing patient privacy is provided. The system includes a first leg assembly extending upwardly. The system further includes a drape support operatively connected at a top end of the first leg assembly and configured such that in an operative position, the drape support extends at least partially across a patient examination table while spaced above the table. The system further includes at least one clip operatively connected to the drape support, each of the at least one clip configured to secure a sheet to the drape support such that the sheet hangs downwardly from the drape support onto the patient to provide privacy to the patient.

### RELATED APPLICATIONS

This application claims priority to U.S. Provisional Patent Application No. 63/513,982, filed Jul. 17, 2023, entitled “Medical Draping System”, hereby incorporated by reference in its entirety.

### FIELD OF THE INVENTION

The present disclosure relates to medical examinations and procedures. More particularly, but not exclusively, the present disclosure relates to a draping system.

### BACKGROUND OF THE ART

For purposes of providing background, problems are generally described in the context of pelvic exams. The pelvic exam can be very uncomfortable for women for a variety of reasons. The procedure itself can cause pain and discomfort. However, anxiousness prior to the exam also contributes to the discomfort associated with a pelvic exam. Several studies have queried women about what aspects of the exam caused anxiety. These studies have demonstrated that privacy and the perception of privacy are very important in making the exam more acceptable to the patient. Several studies have found that anxiety levels increase when there is a perception that there is a lack of body privacy during the exam.

During a pelvic exam, physicians try to drape the pelvis using a sheet, but this is often not successful in truly creating a visual barrier and maintaining the desired level of privacy. In particular, the sheet often gets in the way of placing the speculum or visualizing the cervix through the speculum and is therefore moved negating any privacy afforded by the sheet. In addition, being mindful of the draping and making adjustments can be distracting for both the physician and the patient. Where adjustments to draping are made, this can further result in increasing the amount of time required for the examination.

Although seemingly unrelated without the benefit of this disclosure, during surgical procedures, anesthesia screens or ether screens may be used to reduce the risk of contamination. It is known that sometimes a drape may be positioned between IV stands or poles on opposite sides of the patient to provide such an effect. In addition, various types of anesthesia screens or ether screens exist which may be operating table mounted.

Therefore, what is needed is new and improved devices and methods for preserving privacy during pelvic exams or other types of examinations or procedures.

### SUMMARY

Therefore, it is a primary object, feature, or advantage to improve over the state of the art.

It is a further object, feature, or advantage to provide improved devices and methods for preserving privacy during pelvic exams, other examinations, or surgeries.

It is a still further object, feature, or advantage to provide an easy and convenient means of attaching a drape to afford better privacy for the woman during a pelvic exam.

Another object, feature, or advantage is to allow a physician to complete a pelvic exam successfully while maintaining privacy and the perception of privacy for the patient.

One or more of these and/or other objects, features, or advantages will become apparent from the specification and claims that follow. The present disclosure is not to be limited by or to these objects, features, or advantages. It is to be understood that no single embodiment need provide each and every object, feature, or advantage as different embodiments may provide different objects, features, or advantages.

According to one aspect, a medical draping system for enhancing patient privacy is provided. The system includes a first leg assembly extending upwardly, the first leg assembly comprising a first leg, a first inner tube telescoping upward from the first leg, and a first adapter sleeve for aligning and fitting the first leg with the first inner tube. The system further includes a drape support operatively connected at a top end of the first leg assembly and configured such that in an operative position, the drape support extends at least partially across a table while spaced above the table. The system further includes at least one clip operatively connected to the drape support, each of the at least one clip configured to secure a sheet to the removable arcuate portion such that the sheet hangs downwardly from the drape support onto the patient to provide privacy to the patient. The drape support may include an arcuate portion, the arcuate portion extending at least partially across a table while spaced above the table. The first leg assembly may be free-standing or may be secured to the table which may be an examination, a pelvic examination table having a pair of operatively connected stirrups, or a surgical table.

According to another aspect, a pelvic examination draping system for use with a pelvic examination table including a pair of stirrups extending outwardly therefrom for use during a pelvic examination is provided. The system includes a first base, a second base spaced apart from the first base, a first leg assembly extending upwardly from the first base, a second leg assembly extending upwardly from the second base, a removable arcuate portion extending between a top of the first leg assembly and a top of the second leg assembly, and at least one clip operatively connected to the removable arcuate portion, each of the at least on clip configured to hold a sheet to the removable arcuate portion such that the sheet hangs downwardly from the arcuate portion to provide privacy during the pelvic examination. The first leg assembly may include a first leg, a first inner tube telescoping upward from the first leg, and an adapter sleeve for aligning and fitting the first leg with the first inner tube. The first leg assembly may further include a first adjustment assembly for adjusting extension of the first inner tube from the first leg. The first adjustment mechanism may include a first threaded rod and a first adjustment knob at an end of the first threaded rod wherein the first threaded rod is orthogonal with the first adapter sleeve.

According to another aspect, a pelvic examination draping system includes a pelvic examination table including a pair of stirrups extending outwardly therefrom for use during a pelvic examination. The system further includes a first leg assembly extending upwardly from the pelvic examination table, an arcuate portion extending from a top of the first leg assembly and across at least a portion of the pelvic examination table, and at least one clip operatively connected to the arcuate portion, each of the at least on clip configured to hold a sheet to the arcuate portion such that the sheet hangs downwardly from the arcuate portion to provide privacy during the pelvic examination.

According to another aspect, a pelvic examination draping system includes a pelvic examination table including a pair of stirrups extending outwardly therefrom for use during a pelvic examination. The system further include a first leg assembly extending upwardly from the pelvic examination table, and a drape support extending from a top of the first leg assembly and across at least a portion of the pelvic examination table. There is at least one clip operatively connected to the drape support, each of the at least one clip configured to hold a sheet to the drape support such that the sheet hangs downwardly from the drape to provide privacy during the pelvic examination.

According to another aspect, a pelvic examination draping system includes a pelvic examination table including a pair of stirrups extending outwardly therefrom for use during a pelvic examination, a first leg assembly extending upwardly from the pelvic examination table, and a drape support extending from a top of the first leg assembly and across at least a portion of the pelvic examination table when the pelvic examination table is in an operative position. The system further includes at least one clip operatively connected to the drape support, each of the at least one clip configured to hold a sheet to the drape support such that the sheet hangs downwardly from the drape to provide privacy during the pelvic examination. Each of the at least one clip includes a central C-shape portion with first and second opposite ends with a first tab on the first end and a second tab on the opposite second end extending outwardly therefrom; wherein the central C-shape portion has a curvature configured to conform to the drape support to securely engage the at least one clip to the drape support. The pelvic examination draping system may further include a bracket for securing the first leg assembly to the pelvic examination table. The first leg assembly may be integrally formed with the drape support and wherein the drape support has a free end opposite the first leg assembly. The first leg assembly may be pivotably mounted thereby allowing rotation of the first leg assembly relative to the bracket and the pelvic examination tables such that the drape support is movable from the operative position to a stored position wherein the drape support does not extend across the pelvic examination table. There may be a plurality of openings within the first leg assembly wherein each of the plurality of openings is associated with a different height such that the pelvic examination draping system is height adjustable. The system may further include a retaining pin configured for placement within one of the plurality of openings to secure the leg assembly to the bracket at desired height.

According to another aspect, a medical draping system for enhancing privacy for a patient is provide. The medical draping system includes a first leg assembly extending upwardly, the first leg assembly comprising a first leg, a drape support operatively connected at a top end of the first leg assembly and configured such that in an operative position, the drape support extends at least partially across a table while spaced above the patient examination table, and at least one clip operatively connected to the drape support, each of the at least one clip configured to secure a sheet to the drape support such that the sheet hangs downwardly from the drape support onto the patient to provide privacy to the patient.

### BRIEF DESCRIPTION OF THE DRAWINGS

FIG. 1 is a perspective view of a medical draping system configured to stand on its own.

FIG. 2 is another view of the medical draping system configured to stand on its own with the drape support removed.

FIG. 3 is a side view of the medical draping system.

FIG. 4 is a top view of the medical draping system.

FIG. 5 is a front view of the medical draping system used in conjunction with a table.

FIG. 6 is a side view of the medical draping system used in conjunction with a table upon which a patient is resting.

FIG. 7 is another view of the medical draping system when used in conjunction with a table and where a sheet is connected to the arch of the medical draping system.

FIG. 8 illustrates a clip which may be used to attach a sheet to the arch of the medical draping system.

FIG. 9 is a view of the medical draping system used in conjunction with a pelvic examination table and with a sheet present.

FIG. 10 illustrates clips used to secure a sheet to the medical draping system.

FIG. 11 illustrates the medical draping system in a system which includes a pelvic examination table.

FIG. 12 illustrates the medical draping system from the viewpoint of a patient.

FIG. 13 illustrates the medical draping system which is integrated into a table instead of being free-standing.

FIG. 14 illustrates the table which allows the position of the medical draping system to be easily altered.

FIG. 15 illustrates alternative geometries for the arcuate portion and/or arms.

FIG. 16 illustrates the medical draping system as may be integrated into a table where a single arm is provided.

FIG. 17 further illustrates the medical draping system for integration into a table.

FIG. 18 illustrates an examination table including a single arm medical draping system.

FIG. 19 illustrates a single arm medical draping system on a floor stand.

### DETAILED DESCRIPTION

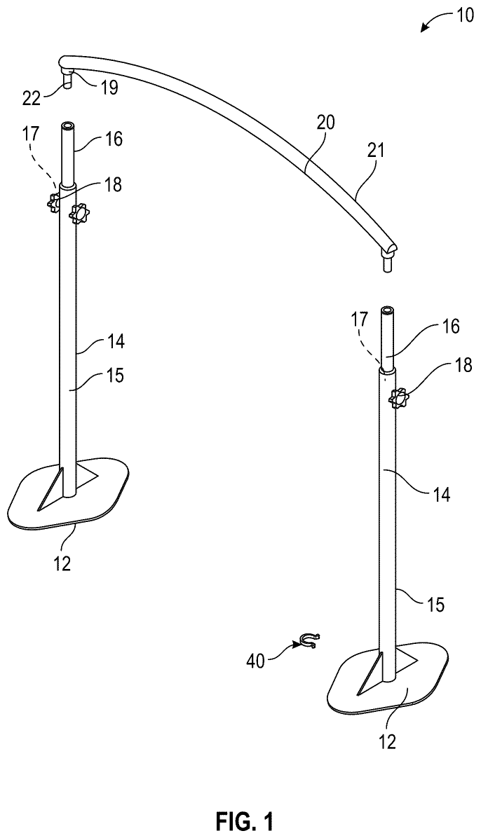

FIG. 1 is a perspective view of a medical draping system 10. The medical draping system 10 shown in FIG. 1 is free-standing. For pelvic exams, the medical draping system 10 may be placed towards the waist of the patient. There are first and second opposite bases 12 which are spaced apart such that they be placed on opposite sides of a bed. Each of the first base and the second base provides support. First and second leg assemblies 14 extend upwardly from their respective bases 12. A drape support 20 is shown which may include an arcuate portion which may extend the length of the drape support 20 or a partial length of the drape support 20. The drape support 20 and in some instances the arcuate portion 21 may extend between the first and second leg assemblies 14.

Each of the first and second leg assemblies 14 may include a leg 15, an inner tube 16 telescoping upward from the leg, and an adapter sleeve 17 for aligning and fitting the leg 15 with the inner tube 16. Each of the leg assemblies 14 may include an adjustment assembly 18 for adjusting extension of the inner tube thereby allowing the drape support 20 to be raised and lowered. Each adjustment assembly 18 may include a threaded road and an adjustment knob at and of the threaded rod. The threaded rod may be positioned orthogonal with the adapter sleeve. Thus, the adjustment knob may be loosened when the drape support 20 is raised or lowered and tightened once the drape support 20 is adjusted to a desired height.

The drape support 20 may have first and second adapters 22 such as at opposite first and second ends of the drape support 20. The adapters 22 allow the drape support to be removably connected to the first and second leg assemblies 14. Each adapter 22 may have a top end and an opposite bottom end. The bottom end may be tapered with a flat portion to securely fit into the leg assembly. The top portion may include a plurality of ribs to encourage a tight fit.

There may be a notch 19 at each of the adapters 22. Each notch 19 may extend outwardly from the adapter 22 to allow for hanging the drape support 20 when not in use. It should be understood that as shown in FIG. 1 the arcuate portion 21 of the drape support 20 extends the entire length of the drape support 20. In addition, the drape support 20 (and thus also it's arcuate portion 21) are removably connected. Thus, in practice, each leg assembly 14 may be positioned on opposite sides of a patient and a table upon which the patient is to be placed. Once the patient is properly positioned on the table, the drape support 20 may be positioned across the patient and a sheet may be secured to the drape support 20 using one or more clip 40 as will be further described later herein.

It is to be understood that various types of materials may be used in the fabrication of the medical draping system 10. For example, the legs 15 may be formed of steel or other durable metal or material. The inner tubes 16 may be performed of aluminum or other lightweight metal. It is also contemplated that various parts of the medical draping system may be formed of plastic including the clip 40. Of course, other materials may be used as may be appropriate for a particular design or its construction.

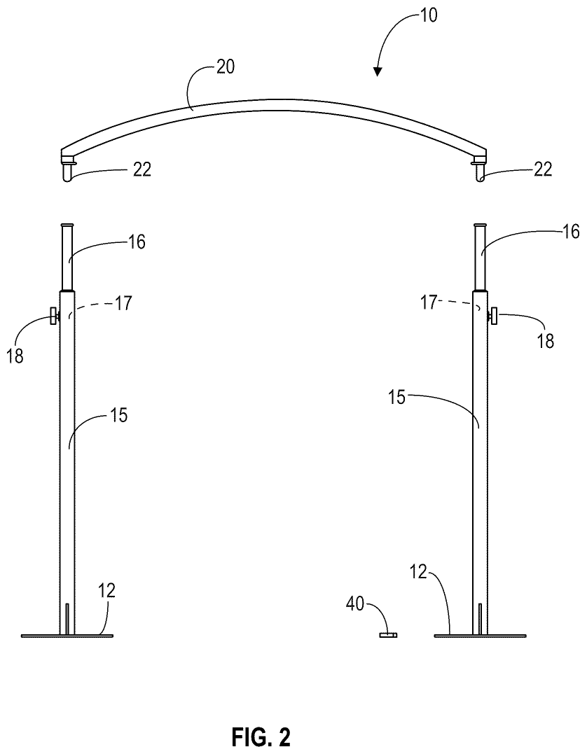

FIG. 2 is another view of the medical draping system 10 configured to stand on its own with the drape support 20 removed. The clip 40 is shown, but in an operative position would be clipped to the drape support 20 to secure a sheet.

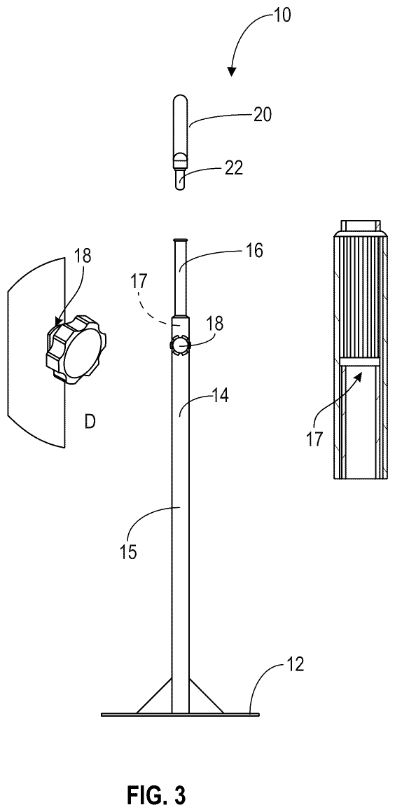

FIG. 3 is a side view of the medical draping system 10. In addition, an adapter sleeve 17 is shown in more detail. The adapter sleeve 17 may be used to align and fit together the inner tube 16 and leg 14. Also shown is the adjustment assembly 18.

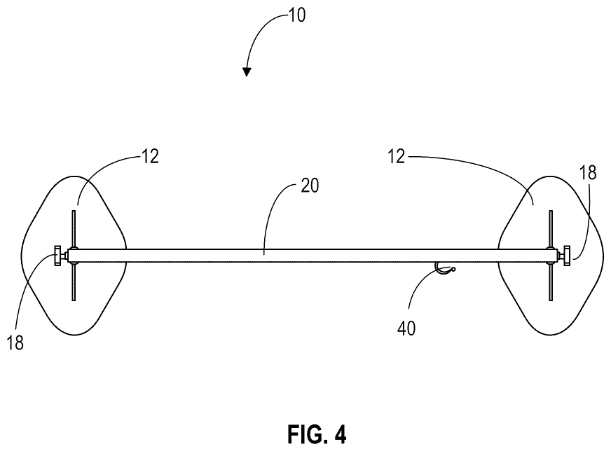

FIG. 4 is a top view of the medical draping system 10 which may include the drape support 20 which may include an arcuate portion which extends across or spans between a first leg and a second leg and the drape support 20 may be positioned above a patient and a table.

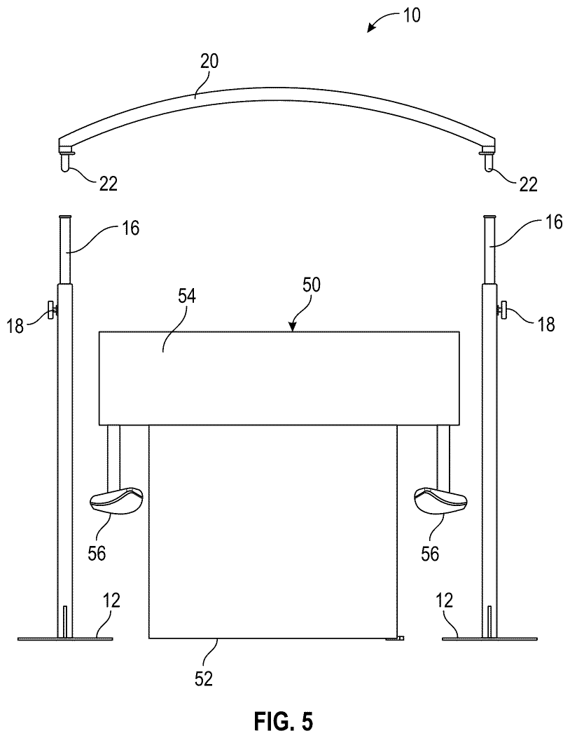

FIG. 5 is a front view of the medical draping system 10 used in conjunction with a table 50. The table 50 may be a pelvic examination table 50 with a support portion 52 and a patient portion 54. The pelvic examination table 50 shown includes a pair of stirrups 56. Other a pelvic examination table 50 is shown, it is contemplated that other types of tables may be used including other types of examination tables and operating tables.

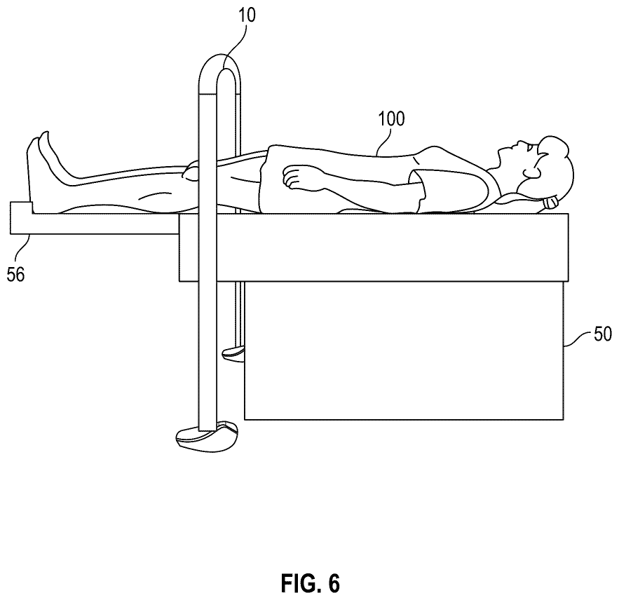

FIG. 6 is a side view of the medical draping system 10 used in conjunction with a table 50 upon which a patient 100 is resting. Note that the table 50 is a pelvic examination table including stirrups 56. The medical draping system 10 is positioned near the pelvic region of the patient 100.

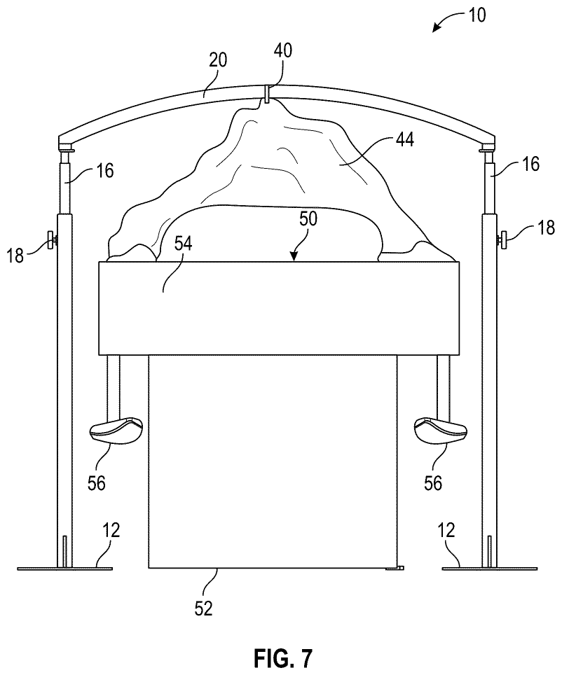

FIG. 7 is another view of the medical draping system 10 when used in conjunction with a table 50 with stirrups 56 and where a sheet 44 is connected to the top of the drape support 20 of the medical draping system 10 using a clip 40. Although a single clip 40 is shown, more than one clip 40 may be used depending upon the desired draping. Generally two clips 40 is enough to allow for desired placement of the sheet 44. The sheet 44 may be draped so that it forms a privacy shield for the patient. Thus, in the case of a pelvic examination, the patient will not be able to see their pelvic region, or the instruments being used by the physician. In some instances, it may be useful to have two clips 40 which are spaced apart and which may allow a patient to better view over the top of the sheet so that they may converse with the physician if necessary while still maintaining a shielded view. Free ends of the sheet 44 may be draped around a patient's legs or otherwise to further enhance privacy and/or the perception of privacy.

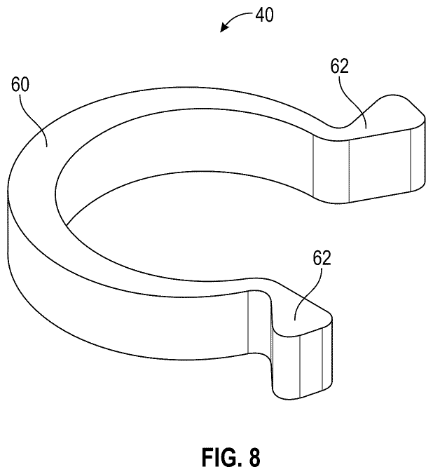

FIG. 8 illustrates a clip 40 which may be used to attach a sheet to the drape support of the medical draping system. As previously explained, although a single clip 40 may be used, more than one clip 40 may be used to secure a sheet. Each clip may include a central C-shape portion 60 which is sized and shaped to securely fit to the drape support. First and second opposite ends 62 are shown each with a tab which allows an individual to better secure the clip 40 to the drape support and to more easily remove the clip 40 from the drape support yet still maintaining a secure fit while connected. The clip 40 may be formed of plastic. Although a particular shape is shown, it is contemplated that other shapes and configurations may be used, provided that the desired functionality is maintained.

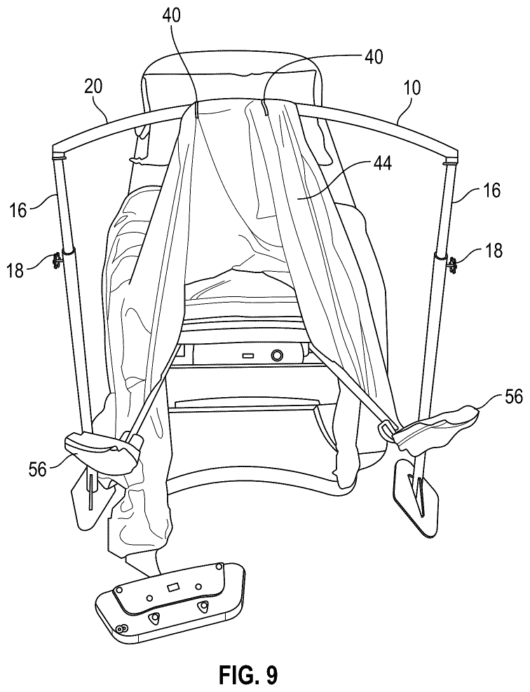

FIG. 9 illustrates the medical draping system used in conjunction with a pelvic examination table and with a sheet present. Note that there are both a first and second clip present and that the examination table is a pelvic examination table with stirrups. Further note the manner in which the sheet is draped which provides a privacy shield for the patient while allowing a physician access.

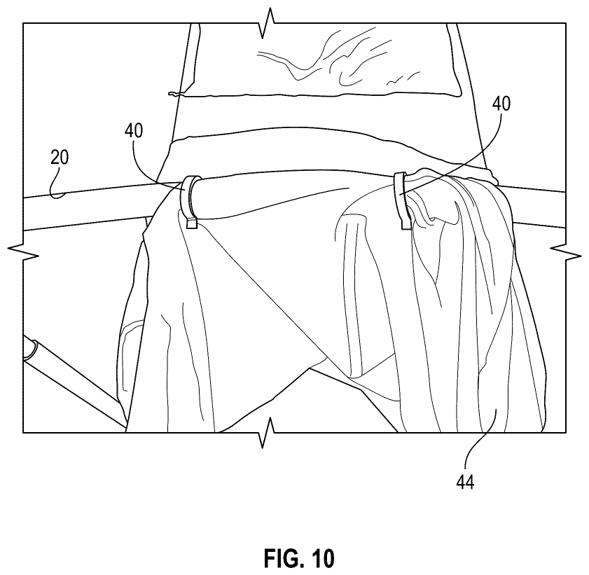

FIG. 10 illustrates clips used to secure a sheet to the medical draping system.

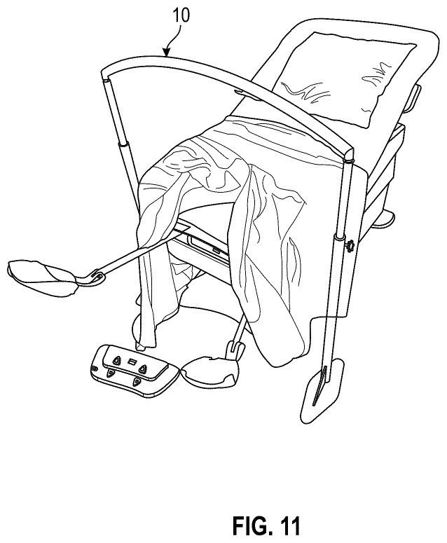

FIG. 11 illustrates the medical draping system where a pelvic examination table is present.

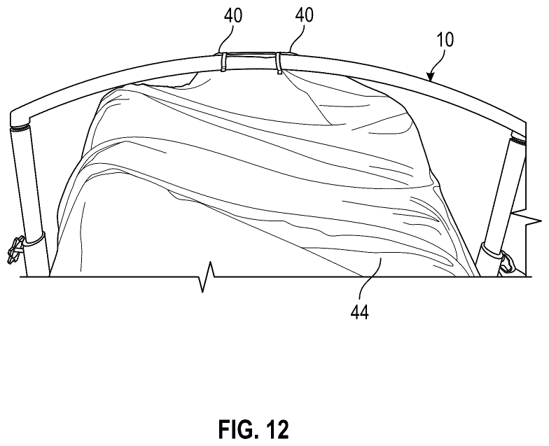

FIG. 12 further illustrates the medical draping system from the viewpoint of a patient. The positioning of the sheet allows for improved privacy and/or perception of privacy from a patient perspective.

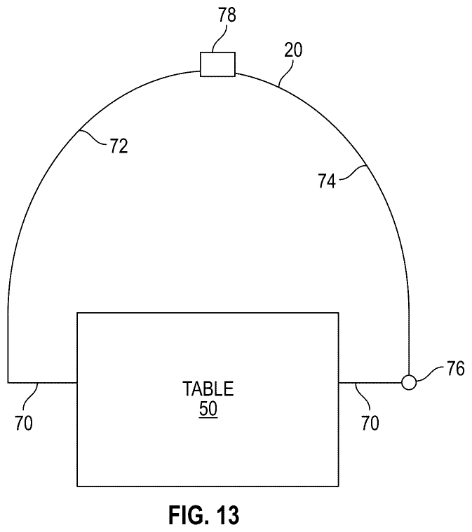

FIG. 13 illustrates the medical draping system which is integrated into a table instead of being free-standing. In some embodiments, instead of the medical draping system being free standing, support members 70 which may also be referred to herein as arms or legs, may extend outwardly from the table and a drape support 20 which may include an arcuate portion extends between the support members 70. In some embodiments, one or more hinges 78 may be present. A connector 76 may also be present. It is contemplated that in the operative position, the drape support 20 may include a first portion 72 and a second portion 74 which may connect at a hinge 78. In addition, one of the portions may be secured to the connector 76. However, to facilitate a patient entering or exiting the table 50, the second portion 74 may be disconnected from the connector 76 and hinged or rotated towards the first portion 72 so that the support 20 does not impeded the patient entering or exiting the table. In some embodiments, there may be connectors 76 on both portions 72, 74 to allow for a patient to enter or exit from either side of the table 50.

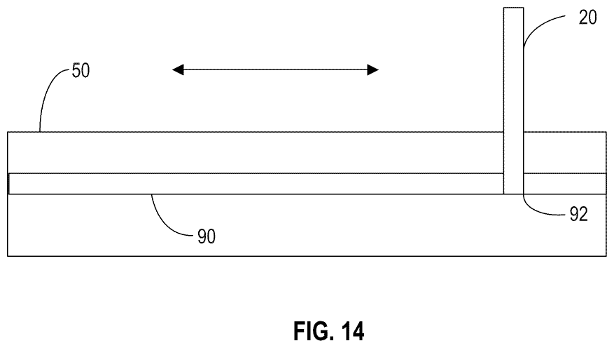

FIG. 14 illustrates the table 50 which allows the position of the drape support 20 to be easily altered. A track 90 may be positioned along the side of the table. The support member or arms or legs associated therewith may engage the track 90. For example, the support member may be operatively connected to a traveler 92 which may slide back and forth along the track. The traveler 92 may include a lock mechanism to secure the traveler 92 in a particular location such as by using a pin or by friction fit. The traveler 92 may include a knob or handle to allow an individual to more easily move the drape support 20 along the table and to lock and unlock the position.

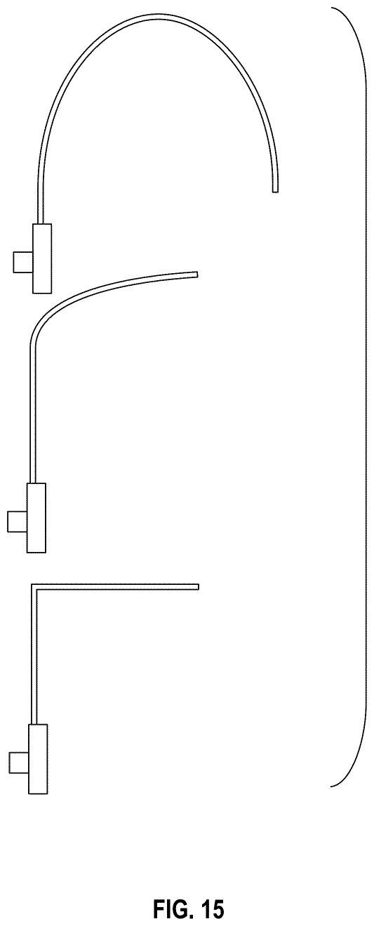

FIG. 15 illustrates alternative geometries for the drape support. In some embodiments, the drape support may have a single leg instead of two. In addition, the drape support may, in some embodiments, not extend completely across the table but a sufficient distance to allow for draping. In the examples shown in FIG. 15, the height of the drape support may be adjusted. Where a single leg is used, it is to be understood that the device may be free standing or may be attached to the table. Where the drape support has one free end, the device may be rotated parallel to the table so that patient can first sit down on the end of the table and then lie back. After she is in position, the device can be rotated 90 degrees so that it is now perpendicular to the length of the table and where used in the context of a pelvic examination table, the drape support would be over the lower abdomen and pelvis of the patient and the sheet may be attached.

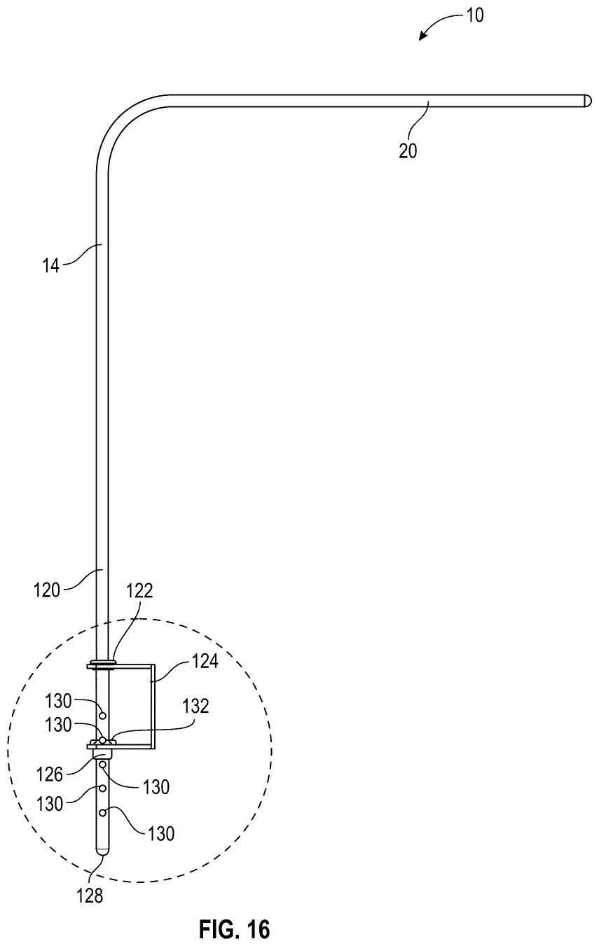

FIG. 16 illustrates an example of a drape system 10 where a single leg assembly 14 is used instead of two and wherein the leg assembly 14 may be attached to a table proximate a bottom end of the leg assembly 14. As shown in FIG. 16 the leg assembly 14 may be formed from a tube 120 which may be a metal tube such as an aluminum tube. The tube 120 may form the leg assembly 14 and be bended to transition from extending generally upward along a side of a table and then inwardly extending over the table to form the drape support 20 such that a sheet or other shielding may be suspended from the drape support 20 to provide a privacy screen. A bracket 124 is shown such as may be attached to a frame of an examination table. It is contemplated that different sizes or shapes of brackets may be used as may be desirable to accommodate a specific examination table. A bushing 122 which may be a plastic pivot bushing is shown above the bracket 124 and a rotation cup 126 which may be a plastic rotation cup is shown below the bracket 124. The leg 14 and integral drape support 20 may thus be rotated so that the drape support 20 may be moved from an operative position where the drape support 20 extends inwardly over the table to a stored position where the drape support is rotated such as along a side of the table or otherwise out of the way so as not to interfere with a patient moving onto or off of the table. This rotation or pivoting action may be provided by alternative means including other pivot mechanisms which allow for rotation of leg assembly 14 relative to the patient table. Where the drape support 10 is not integral with the leg assembly 14, the drape support 10 may be rotated relative to the leg assembly 14 in addition to the patient examination table and the bracket 124 instead of having the leg assembly 14 rotate.

The drape system 10 may be configured to be height adjustable. As shown in FIG. 16, height adjustable functionality may be provided by including a plurality of openings 130 along a length of the tube 120, so that the tube 120 may be positioned at different heights. A retaining pin 132 may be selectively positioned through a desired one of the openings 130 in order to secure and maintain the drape system 10 at a desired height. An end cap 128 may also be inserted into an open bottom end of the tube 120. The end cap 128 may be formed of plastic.

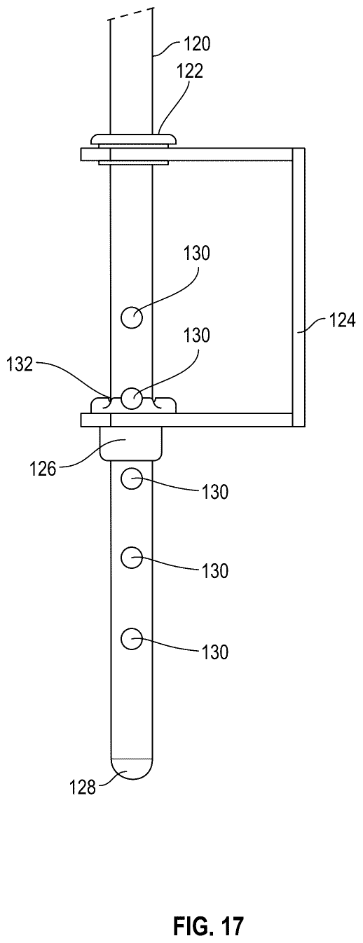

FIG. 17 further illustrates the end portion of the tube 120 and the interface between this portion of the leg assembly and an examination table in more detail. The bracket 124 is shown along with the pivot bushing 122 positioned above the bracket 124 and the plastic rotation cup 126 positioned below the plastic bushing to allow for rotation of the tube 120 which may be a cylindrical tube. A plurality of openings 130 are shown along with a retaining pin 132 which may be selectively positioned in one of the plurality of openings 130 to set the height for the drape support 20. The end cap 128 prevents an open end of the cylindrical tube from being exposed.

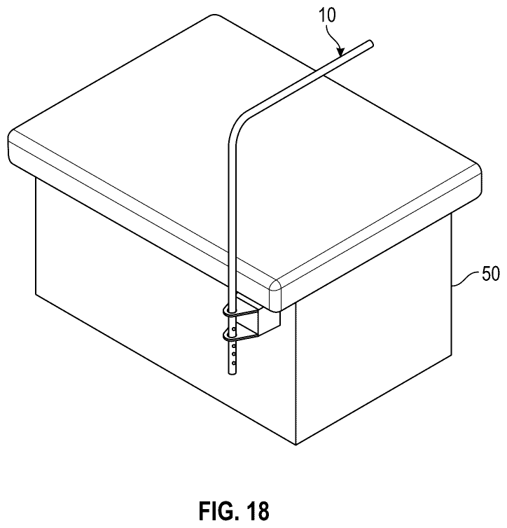

FIG. 18 illustrates the drape support system 10 as shown in FIG. 16 and FIG. 17, for example, attached to an examination table 50 when in an operative position. Note that the drape support system allows a drape support to extend across a width of the table. A drape (not shown) may be attached to one or more clips as has previously been shown and described.

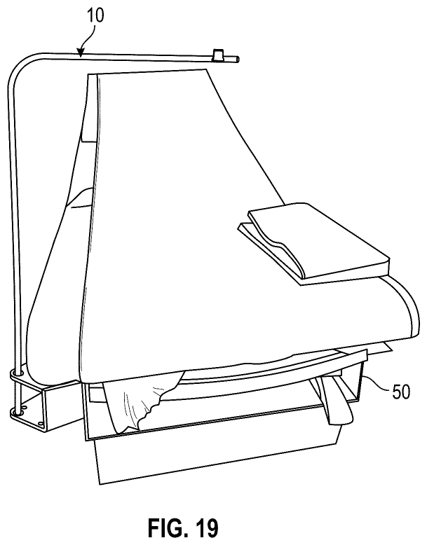

FIG. 19 further illustrates a drape support system 10 when used with an examination table 50. As shown in FIG. 19, the drape support system 10 uses only a single leg assembly similar to that shown in FIG. 18. However, in FIG. 19 instead of the drape support system 10 being attached to the examination table 50, the drape support system 10 is free standing with its own support stand such as previously shown and described. A drape (not shown) may be attached to one or more clips as has previously been shown and described.

Therefore, a medical draping system and related methods and devices have been shown and described. Although specific embodiments are provided, numerous options, variations, and alternatives are contemplated. For example, although specific examples in the context of pelvic examinations and pelvic examination tables have been provided, the medical draping system may be used in other contexts for other types of examination or for other types of surgeries. For example, during Caesarean procedures, the draping allows for privacy separation from the patient and possibly a companion from the activity being performed by an obstetrician. Of course, any number of other examinations and surgical uses are contemplated.

For example, although various embodiments are shown, other variations are contemplated including using the structures associated with ether screens as may be known in the art. Similarly, where attached to a pelvic examination table, it is contemplated that the attachment may be provided in any number of different ways including with brackets of various sizes and shapes. Where the medical draping system allows for the drape support to be rotated, this rotation may be achieved in any number of different ways including through any number of different pivot assemblies. Similarly, where a height adjustment feature is provided, this height adjustment may be provided in any number of different ways including through pressure fit, snap fit, the use of pins, or otherwise.

Therefore, various systems, devices, and methods have been shown and described herein. Although specific embodiments and examples are described, it is to be understood that any number of combinations of features from different embodiments, as well as options, and alternatives are contemplated.

Throughout this specification, plural instances may implement components, operations, or structures described as a single instance. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment and elements from different embodiments may be combined.

As used herein, the terms “comprises”, “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present), and B is false (or not present), A is false (or not present), and B is true (or present), and both A and B are true (or present).

In addition, use of the “a” or “an” are employed to describe elements and components of the embodiments herein. This is done merely for convenience and to give a general sense of the disclosure. This description should be read to include one or at least one and the singular also includes the plural unless it is obvious that it is meant otherwise.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

As used herein, a plurality of items, structural elements, compositional elements, and/or materials may be presented in a common list for convenience. However, these lists should be construed as though each member of the list is individually identified as a separate and unique member. Thus, no individual member of such a list should be construed as a de facto equivalent of any other member of the same list solely based on their presentation in a common group without indications to the contrary.

Reference throughout this specification to “an example” means that a particular feature, structure, or characteristic described in connection with the example is included in at least one embodiment. Thus, appearances of the phrases “in an example” in various places throughout this specification are not necessarily all referring to the same embodiment or example. Features of different examples may be combined.

The invention is not to be limited to the particular embodiments described herein. In particular, the invention contemplates numerous variations in the specific methodology used with respect to the deep manifold learning algorithms. The foregoing description has been presented for purposes of illustration and description. It is not intended to be an exhaustive list or limit any of the invention to the precise forms disclosed. It is contemplated that other alternatives or exemplary aspects are considered included in the invention. The description is merely examples of embodiments, processes, or methods of the invention. It is understood that any other modifications, substitutions, and/or additions can be made, which are within the intended spirit and scope of the invention.

What is claimed is:

1. A medical draping system for enhancing privacy for a patient, the medical draping system comprising:
a first leg assembly extending upwardly, the first leg assembly comprising a first leg;
a drape support operatively connected at a top end of the first leg assembly and configured such that in an operative position, the drape support extends at least partially across a patient examination table while spaced above the patient examination table; and
at least one clip operatively connected to the drape support, each of the at least one clip configured to secure a sheet to the drape support such that the sheet hangs downwardly from the drape support onto the patient to provide privacy to the patient.

2. The medical draping system of claim 1 further comprising:
a first inner tube telescoping upward from the first leg, and a first adapter sleeve for aligning and fitting the first leg with the first inner tube.

3. The medical draping system of claim 2 wherein the drape support comprises an arcuate portion, the arcuate portion extending at least partially across a patient examination table while spaced above the patient examination table.

4. The medical draping system of claim 1 wherein the medical draping system further includes the patient examination table and wherein the first leg assembly is secured to the patient examination table.

5. The medical draping system of claim 4 wherein the patient examination table is a pelvic examination table having a pair of operatively connected stirrups.

6. The medical draping system of claim 2 further comprising a second leg assembly extending upwardly, the second leg assembly comprising a second leg, a second inner tube telescoping upward from the second leg, and a second adapter sleeve for aligning and fitting the second leg with the second inner tube, wherein the second leg assembly is on an opposite side of the patient examination table from the first leg assembly, and wherein in the operative position the drape support is further operatively connected to the second leg assembly.

7. The medical draping system of claim 6 further comprising a first base and a second base spaced apart from the first base and wherein the first base is operatively connected to the first leg assembly at a bottom of the first leg assembly to support the first leg assembly and wherein the second base is operatively connected to the second leg assembly at a bottom of the second leg assembly to support the second leg assembly.

8. The medical draping system of claim 7 wherein the wherein the first leg assembly further comprises a first adjustment assembly for adjusting extension of the first inner tube from the first leg and wherein the second leg assembly further comprises a second adjustment assembly for adjusting extension of a second inner tube from a second leg.

9. The medical draping system of claim 8 wherein the first adjustment assembly comprises a first threaded rod and a first adjustment knob at an end of the first threaded rod wherein the first threaded rod is orthogonal with the first adapter sleeve and wherein the second adjustment assembly comprises a second threaded rod and a second adjustment knob at an end of the second threaded rod wherein the second threaded rod is orthogonal with a second adapter sleeve.

10. The medical draping system of claim 1 wherein each of the at least one clip comprises a central C-shape portion with first and second opposite ends with a first tab on a first end and a second tab on an opposite second end of the clip.

11. A pelvic examination draping system for use with a pelvic examination table including a pair of stirrups extending outwardly therefrom for using during a pelvic examination, the pelvic examination draping system comprising:
a first base;
a second base spaced apart from the first base;
a first leg assembly extending upwardly from the first base;
a second leg assembly extending upwardly from the second base;
a removable arcuate portion extending between a top of the first leg assembly and a top of the second leg assembly; and
at least one clip operatively connected to the removable arcuate portion, each of the at least one clip configured to hold a sheet to the removable arcuate portion such that the sheet hangs downwardly from the removable arcuate portion to provide privacy during the pelvic examination.

12. The pelvic examination draping system of claim 11 wherein the first leg assembly comprises a first leg, a first inner tube telescoping upward from the first leg, and a first adapter sleeve for aligning and fitting the first leg with the first inner tube.

13. The pelvic examination draping system of claim 12 wherein the first leg assembly further comprises a first adjustment assembly for adjusting extension of the first inner tube from the first leg.

14. The pelvic examination draping system of claim 13 wherein the first adjustment assembly comprises a first threaded rod and a first adjustment knob at an end of the first threaded rod wherein the first threaded rod is orthogonal with the first adapter sleeve.

15. The pelvic examination draping system of claim 11 further comprising a first adapter positioned on a first end of the removable arcuate portion for connecting the first end of the arcuate portion to the first leg assembly and a second adapter positioned on an opposite second end of the removable arcuate portion for connecting the opposite second end of the arcuate portion to the second leg assembly.

16. The pelvic examination draping system of claim 15 wherein the first adapter has a top end and an opposite bottom end and wherein the opposite bottom end is tapered with a flat portion to securely fit into the first leg assembly and wherein the top end includes a plurality of ribs.

17. The pelvic examination draping system of claim 16 further comprising a notch extending outwardly from the first adapter to allow for hanging the removable arcuate portion when not in use.

18. The pelvic examination draping system of claim 11 wherein each of the at least one clip comprises a central C-shaped portion with first and second opposite ends with a first tab on a first end of the central C-shaped portion and a second tab on the opposite second end of the central C-shaped portion.

19. A pelvic examination draping system comprising:
a pelvic examination table including a pair of stirrups extending outwardly therefrom for use during a pelvic examination;
a first leg assembly extending upwardly from the pelvic examination table;
a drape support extending from a top of the first leg assembly and across at least a portion of the pelvic examination table when the pelvic examination table is in an operative position;
at least one clip operatively connected to the drape support, each of the at least one clip configured to hold a sheet to the drape support such that the sheet hangs downwardly from the drape to provide privacy during the pelvic examination;
wherein each of the at least one clip comprises a central C-shape portion with first and second opposite ends with a first tab on the first end and a second tab on the opposite second end extending outwardly therefrom; and
wherein the central C-shape portion has a curvature configured to conform to the drape support to securely engage the at least one clip to the drape support.

20. The pelvic examination draping system of claim 19 further comprising:
a bracket for securing the first leg assembly to the pelvic examination table;
wherein the first leg assembly is integrally formed with the drape support and wherein the drape support has a free end opposite the first leg assembly;
wherein the first leg assembly is pivotably mounted thereby allowing rotation of the first leg assembly relative to the bracket and the pelvic examination table such that the drape support is movable from the operative position to a stored position wherein the drape support does not extend across the pelvic examination table;
a plurality of openings within the first leg assembly wherein each of the plurality of openings is associated with a different height such that the pelvic examination draping system is height adjustable; and
a retaining pin configured for placement within one of the plurality of openings to secure the leg assembly to the bracket at desired height.
